(19) United States
(12) Patent Application Publication
(10) Pub. No.: US 2008/0194929 A1
(43) Pub. Date: Aug. 14, 2008

(54) Photoacoustic Assay Method and Apparatus

(75) Inventors: Benny Pesach, Rosh-Ha'ayin (IL); Gabriel Bitton, Jerusalem (IL); Michal Balberg, Jerusalem (IL)

Correspondence Address: PRTSI, P.O. Box 16446, Arlington, VA, 22215, US

(73) Assignee: Glucon, Inc., Boulder, CO (US)

(21) Appl. No.: 10/551,543

(22) PCT Filed: Mar. 29, 2004
(86) PCT No.: PCT/IL04/00289

§ 371 Date: Aug. 8, 2006

Related U.S. Application Data
(60) Provisional application No. 60/458,973, filed on Apr. 1, 2003.

(51) Int. Cl. A61B 5/1455; A61B 8/00

(52) U.S. Cl. 600/310; 600/437

(57) ABSTRACT

Apparatus (20, 100) for assaying a target analyte in a localized tissue region (22) that may include the target and other analytes comprising: a light source (34, 104) that illuminates the region with light at each of a plurality of wavelengths at which light is absorbed and/or scattered by tissue in the region wherein light at a least one of the wavelengths is absorbed or scattered by the target analyte; a signal generator (40) that generates signals responsive to intensity of the light from the light source (34, 104) at different locations in the localized region (22); and a controller (32, 102) that: receives the generated signals; processes the signals to determine an extinction coefficient for light in the localized region at each wavelength; and determines the concentration of the target analyte responsive to a solution of a set of simultaneous equations having as unknown variables concentrations of a plurality of analytes in the region (22), one of which is the target analyte, wherein each equation in the set expresses a relationship between the extinction coefficient, the absorption coefficient and/or the reduced scattering coefficient for light at a different one of the plurality of wavelengths and at least one of the equations expresses a relationship between the extinction coefficient and the reduced scattering coefficient.

### FIELD OF THE INVENTION

The invention relates to non-invasive in-vivo methods and apparatus for determining the concentration of a substance in a body.

### BACKGROUND OF THE INVENTION

Non-invasive methods for assaying a “target” analyte, such as for example glucose, comprised in a region of body tissue are known in the art. In a near infrared spectroscopy (NIRS) method, light at a plurality of different wavelengths in a near infrared band of wavelengths is transmitted into a tissue region of the body to assay a target analyte in the tissue region. Light at least one of the wavelengths, a “target wavelength” is absorbed or scattered by the target analyte. Intensity of light at the different wavelengths that is transmitted through the tissue region or scattered out of the tissue region is measured. The measured intensities are used to isolate and determine the contribution of the target analyte to an absorption or scattering coefficient of the tissue region at the target wavelength in the presence of contributions to the absorption or scattering coefficient by other “interfering” analytes in the tissue. Known values for the absorption or scattering cross sections of the target analyte at the target wavelength and the determined contribution of the target analyte to the absorption or scattering component are used to assay the target component in the tissue.

However, NIRS methods provide concentration measurements of a target analyte in tissue that are averages over relatively long optical path lengths through the tissue of light used to acquire the measurements. As a result, NIRS methods and technologies generally suffer from poor spatial resolution. In addition, NIRS signals tend to suffer from noise generated by scattering of light at tissue interfaces, such as the skin, and tissue inhomogeneities. NIRS methods tend therefore to exhibit relatively poor signal to noise ratios.

An article by G. Yoon, et al. “Determination Of Glucose Concentration in a Scattering Medium Based on Selected Wavelengths by Use Of an Overtone Absorption Band”, in APPLIED OPTICS 1 Mar. 2002; Vol. 41, No 7 describes an NIRS method and device for assaying glucose in a tissue medium. The method describes criteria for choosing discrete wavelengths for light used in performing an NIRS assay so as to reduce influence of interfering analytes in the tissue on the results of the assay. Whereas the method is based on measuring NIRS absorption spectra of the tissue medium, the tissue medium is assumed to be scattering as well as absorbing. The article describes a device for assaying glucose having a light source and a detector that are used to measure absorption spectra for the medium that have their relative positions optimized so that “measured spectra can be independent of medium scattering”.

For many medical procedures it is advantageous to accurately determine concentration of a target analyte for tissue regions that are relatively spatially localized. For example, in assaying glucose levels for a patient it is generally advantageous to measure glucose levels in blood. To acquire such measurements, the measurements should be spatially localized to a blood vessel or blood vessels so that the measurements are not “diluted”, for example, by glucose levels in interstitial fluids. NIRS methods and devices, because of their relatively poor spatial resolution generally cannot provide such localized assays.

Methods of measuring concentration of a target analyte in a tissue region using a time-resolved photoacoustic effect or optical coherence tomography (OCT) can provide measurements resolved to a relatively high spatial resolution.

In a method using a time resolved photoacoustic effect, light at least one wavelength for which light is absorbed or scattered by the target analyte is used to generate photoacoustic waves in the tissue region. Pressure produced by acoustic energy from the photoacoustic waves that arrives at a suitable acoustic transducer or transducers is used to assay the target analyte at locations in the region at which the photoacoustic waves are generated. Locations at which the photoacoustic waves are generated can be determined to within about 10 microns axially, along a direction of propagation of the waves, and to within about 200 microns laterally. As a result, an assay of the target analyte can be spatially localized to relatively small volumes having axial dimensions of about 10 microns and lateral dimensions of about 200 microns.

In OCT, light from a semi-coherent light source comprised in an interferometer is split into a reference light beam and a light beam that illuminates the tissue region. Light from the reference beam is reflected from a mirror to an “interference region” in the interferometer where it interferes with light scattered from the tissue region that reaches the interference region. An interference signal in the interference region is generated substantially only for reference and scattered light that reach the interference region after traveling substantially equal optical path lengths. As a result, the interference signal is generated substantially only for light scattered from material located in a small volume of the tissue region for which the optical path lengths of scattered light and reference light are substantially equal. The amplitude of the interference signal is substantially proportional to a scattering coefficient for material in the small volume and is used to assay the target analyte in the small volume. Optical coherence tomography can provide axial spatial resolution of about a micron and lateral resolution of about 3 microns. Spatial resolution of an assay provided by OCT assaying is therefore on the order of a small number of microns.

However, absorption and scattering cross sections of a target and interfering analytes in a tissue region contribute to photoacoustic or OCT signals used to assay the target analyte. Accuracy of the assay is generally compromised if contributions to the signals from scattering cross sections of the analytes are not assessed and distinguished from contributions to the signals from absorption cross sections of the analytes. Prior art has not provided methods for assaying a target analyte in a tissue region responsive to photoacoustic or OCT signals for which scattering cross section contributions to the signals are assessed and distinguished from absorption cross section contributions to the signals.

### SUMMARY OF THE INVENTION

An aspect of some embodiments of the present invention relates to providing assay apparatus that uses the photoacoustic effect to assay a target analyte in a spatially localized tissue region and accounts for scattering of light used to generate the photoacoustic effect in determining the assay.

An aspect of some embodiments of the present invention relates to providing assay apparatus that uses OCT signals to assay a target analyte in a spatially localized tissue region and accounts for scattering of light used to generate the OCT signals in determining the assay.

An aspect of some embodiments of the present invention relates to providing a method for incorporating the effects of scattering of light in assaying a target analyte in a tissue region using the photoacoustic effect and/or OCT.

An assay apparatus in accordance with an embodiment of the present invention comprises at least one light source that illuminates the tissue region with light at each of a plurality of different wavelengths, hereinafter referred to as “mensuration wavelengths”. For at least one of the mensuration wavelengths light is absorbed and/or scattered, optionally strongly, by the target analyte.

In some embodiments of the invention, the assay apparatus comprises at least one acoustic transducer, which senses pressure in photoacoustic waves generated at different locations in the tissue region responsive to the light. Alternatively or additionally, the assay apparatus comprises an OCT interferometer. Interference signals are generated by the interferometer between light scattered from material at different locations in the tissue region and a reference beam of light provided by the light source. An extinction coefficient for each mensuration wavelength is determined for the tissue region either responsive to signals generated by the at least one acoustic transducer or interference signals generated by the interferometer.

The mensuration wavelengths are determined so that each extinction coefficient is dependent on concentration of at least one of a same plurality of “mensuration” analytes. One of the mensuration analytes is the target analyte and the remaining mensuration analytes are “interfering” analytes. Each extinction coefficient therefore defines an equation having as an unknown variable a concentration of at least one of the mensuration analytes. Together, the extinction coefficients define a plurality of simultaneous equations having as unknown variables concentrations of the mensuration analytes in the tissue region. The number of the plurality of mensuration wavelengths and therefore the number of simultaneous equations is equal to or greater than the number of the plurality of mensuration analytes.

In accordance with an embodiment of the present invention, a wavelength dependent function, hereinafter a “scattering coefficient function”, which is parameterized by at least one characteristic parameter, is used to provide a value for the scattering coefficient for at least one of the mensuration wavelengths that contributes to the extinction coefficient at the wavelength. The equation defined by the extinction coefficient for a given mensuration wavelength comprises a term which is the scattering coefficient function evaluated at the mensuration wavelength. An assay of the target analyte is provided responsive to a solution of the simultaneous equations.

In some embodiments of the present invention, at least one characteristic parameter of the scattering coefficient function is determined from information extraneous to information used to determine the set of simultaneous equations. In some embodiments of the present invention, at least one characteristic parameter of the scattering coefficient function is determined from an extinction coefficient determined from signals provided by the at least one acoustic transducer or alternatively by the interferometer.

In some embodiments of the present invention, the number of the plurality of mensuration wavelengths and therefore extinction coefficients is greater than the number of the plurality of mensuration analytes and the simultaneous equations are used to determine at least one characteristic parameter of the scattering function.

In some embodiments of the invention, the scattering coefficient function is determined assuming that optical scattering in the tissue region is Mie scattering.

In some embodiments of the invention, the target analyte is glucose and an assay apparatus is used to provide in vivo measurements of glucose in a blood vessel in the body of a patient.

There is therefore provided in accordance with an embodiment of the present invention apparatus for assaying a target analyte in a localized tissue region that may include the target and other analytes comprising: a light source that illuminates the region with light at each of a plurality of wavelengths at which light is absorbed and/or scattered by tissue in the region wherein light at least one of the wavelengths is absorbed or scattered by the target analyte; a signal generator that generates signals responsive to intensity of the light from the light source at different locations in the localized region; and a controller that: receives the generated signals; processes the signals to determine an extinction coefficient for light in the localized region at each wavelength; and determines the concentration of the target analyte responsive to a solution of a set of simultaneous equations having as unknown variables concentrations of a plurality of analytes in the region, one of which is the target analyte, wherein each equation in the set expresses a relationship between the extinction coefficient, the absorption coefficient and/or the reduced scattering coefficient for light at a different one of the plurality of wavelengths and at least one of the equations expresses a relationship between the extinction coefficient and the reduced scattering coefficient.

Optionally, the at least one equation that expresses a relationship between the extinction coefficient and the reduced scattering coefficient includes a dependence on the absorption coefficient.

Additionally or alternatively the reduced scattering coefficient at least one of the wavelengths is a measured value of the reduced scattering coefficient.

In some embodiments of the present invention, the reduced scattering coefficient at least one of the wavelengths is a value determined responsive to an analytic expression.

In some embodiments of the present invention, the reduced scattering coefficient at least one of the wavelengths is expressed as an analytic function. Optionally, the analytic expression is a function of at least one unknown variable having a value determinable responsive to a solution of the simultaneous equations. Optionally, the at least one unknown variable is a concentration of at least one of the target analyte and the other analytes.

In some embodiments of the present invention, the function comprises an expression of the form Bλ^{-C} where λ represents the wavelength and B and C are constants.

In some embodiments of the present invention, the signal generator comprises at least one acoustic transducer that generates signals responsive to acoustic energy that reaches the transducer from photoacoustic waves generated in the region by the light.

In some embodiments of the present invention, the signal generator comprises an optical coherence tomography device that receives light from the light source that is scattered from the region and generates an interference signal responsive to an interference pattern between the scattered light and light from the light source reflected by a reflector.

In some embodiments of the present invention, the controller identifies and locates the localized region in a larger region comprising the localized region.

Optionally, to identify and locate the localized region the controller: controls the light source to illuminate the larger region with light that is absorbed by a component characteristic of the localized region; receives signals generated by the signal generator responsive to intensity of the light from the light source in different locations in the larger region; uses the signals to assay the characteristic component in different localized regions in the larger region; and identifies and locates the localized region responsive to the assay.

Optionally, the apparatus comprises at least one acoustic transducer controllable to transmit ultrasound, and to identify and locate the localized region the controller: controls the at least one transducer to transmit ultrasound into the larger region; receives signals generated by the at least one acoustic transducer responsive to acoustic energy reflected by features in the larger region from the transmitted ultrasound; and uses the signals to identify and locate the features and thereby the localized region.

In some embodiments of the present invention, the localized region is a bolus of blood.

There is further provided in accordance with the present invention a method of assaying a target analyte in a region of body tissue that may include the target and other analytes comprising: determining an extinction coefficient for light at each of a plurality of different wavelengths at which light is absorbed and/or scattered by tissue in the region and wherein light at least one of the wavelengths is absorbed and/or scattered by the analyte; providing a value or an analytic expression for the reduced scattering coefficient at each wavelength; and determining the concentration of the target analyte responsive to a solution of a set of simultaneous equations having as unknown variables concentrations of a plurality of analytes in the region, one of which is the target analyte, wherein each equation in the set expresses a relationship between the extinction coefficient, the absorption coefficient and/or the reduced scattering coefficient for light at a different one of the plurality of wavelengths and at least one of the equations expresses a relationship between the extinction coefficient and the reduced scattering coefficient.

Optionally, the at least one equation that expresses a relationship between the extinction coefficient and the reduced scattering coefficient includes a dependence on the absorption coefficient.

Additionally or alternatively determining the extinction coefficient at least one of the wavelengths of the plurality of wavelengths optionally comprises: from a given location illuminating the region with light at the wavelength so as to generate photoacoustic waves in the region; determining a rate of decrease amplitude of the generated photoacoustic waves with increase of distance in the tissue region from the given location; and determining the extinction coefficient from the determined rate of decrease.

In some embodiments of the present invention, determining the extinction coefficient at least one of the wavelengths of the plurality of wavelengths comprises: from a given location illuminating the region with light at the wavelength; using optical coherence tomography to determine a rate of decrease of intensity of the light with increase of distance in the tissue region from the given location; and determining the extinction coefficient from the determined rate of decrease.

In some embodiments of the present invention, the reduced scattering coefficient at least one of the wavelengths is a measured value of the reduced scattering coefficient.

In some embodiments of the present invention, the reduced scattering coefficient at least one of the wavelengths is a value determined responsive to an analytic expression.

In some embodiments of the present invention, the method comprises expressing the reduced scattering coefficient in at least one of the equations as an analytic function.

In some embodiments of the present invention, the analytic expression is a function of at least one unknown variable having a value determinable responsive to a solution of the simultaneous equations. Optionally, the at least one unknown variable is a concentration of at least one of the target analyte and other analytes.

In some embodiments of the present invention, the analytic expression comprises an expression of the form Bλ^{-C} where λ represents the wavelength and B and C are constants.

In some embodiments of the present invention, the method comprises identifying and locating the localized region in a larger region comprising the localized region.

Optionally, identifying and locating the localized region comprises: illuminating the larger region with light that is absorbed by a component characteristic of the localized region; generating signals responsive to intensity of the light at different locations in the larger region; using the signals to assay the characteristic component in different localized regions in the larger region; and identifying and locating the localized region responsive to the assay.

Additionally or alternatively, identifying and locating the localized region comprises: transmitting ultrasound into the larger region; generating signals responsive to acoustic energy reflected by features in the larger region from the transmitted ultrasound; and using the signals to identify and locate the features; using the identities and locations of the features to identify and locate the localized region.

In some embodiments of the present invention, the localized region is a bolus of blood.

### BRIEF DESCRIPTION OF FIGURES

Non-limiting examples of embodiments of the present invention are described below with reference to figures attached hereto, which are listed following this paragraph. In the figures, identical structures, elements or parts that appear in more than one figure are generally labeled with a same numeral in all the figures in which they appear. Dimensions of components and features shown in the figures are chosen for convenience and clarity of presentation and are not necessarily shown to scale.

FIG. 1 schematically shows an assay apparatus assaying glucose using the photoacoustic effect, in accordance with an embodiment of the present invention; and

FIG. 2 schematically shows an assay apparatus that uses both the photoacoustic effect and OCT to assay glucose, in accordance with an embodiment of the present invention.

### DETAILED DESCRIPTION OF PREFERRED EMBODIMENTS

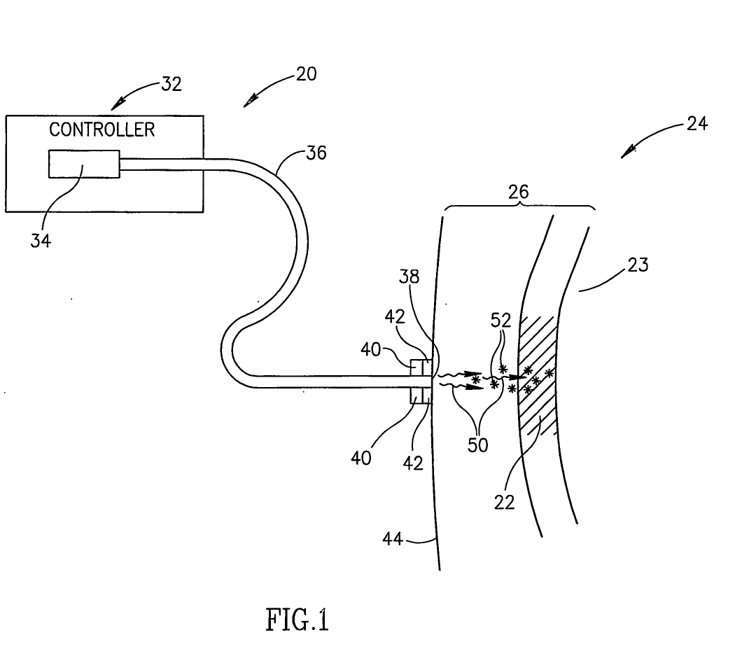

FIG. 1 schematically shows an assay apparatus 20, hereinafter referred to as a “glucometer”, assaying glucose in a “target region” 22 of a body part 24 of a patient, in accordance with an embodiment of the invention. Target region 22 is optionally located in a region 26 of soft tissue of body part 24 and comprises a body fluid, such as for example interstitial fluid, having a concentration of glucose. Optionally, target region 22 is a volume of body fluid having a concentration of glucose and region 26 is a region of a fluid cavity containing the body fluid. For example, as in FIG. 1, target region 22 is a bolus of blood and the fluid cavity a blood vessel 23.

Glucometer 20 optionally comprises a controller 32, a light source 34, optionally located in the controller, and an optic fiber 36 coupled to the light source. An end 38 of fiber 36 is optionally mounted to a support structure 40, hereinafter a “probe head”, to which an acoustic transducer or array of transducers is mounted Any of various appropriate acoustic transducers or array of transducers may be used in the practice of the invention. By way of example, in FIG. 1 probe head 40 has an array of acoustic transducers 42 positioned circumferentially around end 38 of optic fiber 36. Only two transducers of the array are shown. Probe head 40 is pressed to skin 44 of body part 24 to position end 38 of fiber 36 close to or contiguous with the body part and to acoustically couple acoustic transducers 42 to the body part.

To assay glucose in blood bolus 22 controller 32, optionally first, controls glucometer 20 to locate blood vessel 23 and the bolus using any of various methods known in the art, such as methods described in PCT publication WO 02/15776, the disclosure of which is incorporated herein by reference. For example, to locate blood bolus 22 controller 32 may control transducers 42 to radiate ultrasound into region 26. Controller 32 processes signals generated by transducers 42 responsive to reflections of the radiated ultrasound from structures in region 26 to determine location of blood vessel 23. Alternatively, controller 32 may control light source 34 to illuminate tissue region 26 with light that is relatively strongly absorbed by blood. Since the light is strongly absorbed by blood, photoacoustic waves are preferentially generated in blood vessel 23. Controller 32 processes signals generated by transducers 42 responsive to acoustic energy from the photoacoustic waves to image features in region 26 and locate blood vessel 23.

In accordance with an embodiment of the invention, controller 32 then controls light source 34 to illuminate region 26 with at least one light pulse, represented by wavy arrows 50, at each of a plurality of N_{λ} mensuration wavelengths λᵢ. The index i indicates a particular one of the N_{λ} mensuration wavelengths and satisfies the condition 1≦i≦N_{λ}.

The at least one pulse of light 50 at target wavelength λᵢ stimulates photoacoustic waves, schematically represented by starbursts 52, in tissue region 26 and bolus 22. Transducers 42 generate signals responsive to pressure in acoustic energy from photoacoustic waves 52 that reach the transducers. The signals are transmitted to controller 32, which processes the signals in accordance with an embodiment of the invention, as described below, to determine glucose concentration in target region 22.

In some embodiments of the invention, the at least one pulse of light 50 transmitted at different mensuration wavelengths λᵢ is transmitted at different times to illuminate bolus 22. In some embodiments of the invention the at least one pulse 50 comprises a train of pulses. In some embodiments of the invention the pulses in the train of light pulses at different mensuration wavelengths λᵢ are transmitted at different pulse repetition rates. Optionally, light pulse trains at different mensuration wavelengths are transmitted simultaneously. Signals generated by acoustic transducers 42 responsive to photoacoustic waves 52 that are stimulated by light pulse trains at different mensuration wavelengths are distinguished using signal processing techniques known in the art, such as appropriate heterodyning and phase locking techniques.

Let intensity of a light pulse 50 transmitted at a mensuration wavelength λᵢ into body part 24, at a distance d from end 38 of fiber 36 be represented by I(λᵢ,d). Assuming that d is larger than the mean free path for photons at wavelength λᵢ, I(λᵢ,d) may be written,

I(λᵢ,d)=Iₒ(λᵢ)exp(−αₑ(λᵢ)d) (1)

where αₑ(λᵢ) is an extinction coefficient in the tissue of the body part for light at wavelength λᵢ and Iₒ(λᵢ) is intensity of light in the light pulse at end 38 of fiber 36. The extinction coefficient is a function of an absorption coefficient αₐ(λᵢ) and a scattering coefficient αₛ(λᵢ) in the tissue for light at the wavelength λᵢ. Under the assumption of the diffusion approximation, the extinction coefficient may be written

αₑ(λᵢ)=[3αₐ(λᵢ)(αₐ(λᵢ)+α′ₛ(λᵢ)]^{1/2} (2)

where,

α'ₛ(λᵢ)=(1−g)αₛ(λᵢ). (3)

α'ₛ(λᵢ) is referred to as a reduced scattering coefficient and g is an anisotropy factor.

Equation (2) may be rearranged to provide an expression for the absorption coefficient αₐ(λᵢ)

αₐ(λᵢ)=½{−α'ₛ(λᵢ)+[α'ₛ(λᵢ)²+( 4/3)αₑ(λᵢ)²]^{1/2}}. (4)

At each wavelength λᵢ, the absorption coefficient αₐ(λᵢ) may be expressed as a sum of absorption coefficients of analytes in region 24 that absorb light at the wavelength λᵢ. The absorption coefficient of a given analyte is a product of an absorption cross-section of the analyte for light at wavelength λᵢ and concentration of the analyte in the body. Let the absorption cross-section of a “j-th” analyte at wavelength λᵢ be represented by σⱼ(λᵢ) and its concentration in blood bolus 22 by xⱼ. In accordance with an embodiment of the present invention the N_{λ} mensuration wavelengths are chosen so that at each mensuration wavelength λᵢ, substantially only at least one of a same plurality of “N_{A}” mensuration analytes contributes to αₐ(λᵢ). One of the N_{A} mensuration analytes σⱼ(λᵢ) is glucose, the target analyte, and one of the N_{λ} mensuration wavelengths λᵢ is a target wavelength corresponding to the target analyte glucose for which target wavelength light is, optionally, strongly absorbed by glucose. The absorption cross section σⱼ(λᵢ) for j=1 and the wavelength λᵢ for i=1 are arbitrarily assigned to represent respectively the absorption cross section for the target analyte glucose and the corresponding target wavelength. The absorption coefficient at wavelength λᵢ may therefore be written,

$$\begin{matrix}{α}_{a} ( {λ}_{i} ) = {\sum }_{j}^{{N}_{A}} {σ}_{j} ( {λ}_{i} ) {x}_{j} \\ = 1 / 2 \{ {α}_{s}^{′} ( {λ}_{i} ) + {[ {{α}_{s}^{′} ( {λ}_{i} )}^{2} + ( 4 / 3 ) {{α}_{e} ( {λ}_{i} )}^{2} ]}^{1 / 2} \} .\end{matrix} \qquad ( 5 )$$

The N_{λ} mensuration wavelengths provide a set of N_{λ} linear equations of the form of equation (5) in the N_{A} unknown concentrations xⱼ (1≦j≦N_{A}). The equations can be solved for any and all the mensuration analyte concentrations xⱼ, and in particular for concentration x₁ of glucose in blood bolus 22 if N_{λ}≧N_{A} and for each mensuration wavelength λ, of the N_{λ} wavelengths the extinction coefficient αₑ(λᵢ) and reduced scattering coefficient α'ₛ(λᵢ) are known.

To determine the extinction coefficient αₑ(λᵢ) for blood bolus 22 for each mensuration wavelengths λᵢ in accordance with an embodiment of the invention, controller 32 processes signals that transducers 42 generate responsive to pressure produced by photoacoustic waves 52 at the transducers.

Pressure sensed by acoustic sensors 42 responsive to photoacoustic waves 52 is time dependent. Pressure sensed at a time “t” following a time at which a light pulse 50 illuminates body part 24 arises from photoacoustic waves generated at locations in the body part for which distance “d” from acoustic sensors 42 is substantially equal to vt, where v is the speed of sound. (The transmission time of the light is negligible.) Let the pressure sensed by acoustic sensors 42 at time t responsive to a pulse of light 50 at wavelength λᵢ that illuminates tissue region 26 be represented by P(λᵢ,t). Then for photoacoustic waves generated at locations in tissue region 26 at a distance d from transducers 42, P (λ_{τ},t) can be written:

P(λᵢ,t)=P(λᵢ,d/v)=Kαₐ(λᵢ)I(λᵢ,d) (6)

where K is a constant of proportionality.
Using equation 1, equation (6) may be rewritten,

P(λᵢ,t)=P(λᵢ,d/v)=Kαₐ(λᵢ) {Iₒ(λᵢ)exp(−αₑ(λᵢ)d)}. (7)

From the time dependence of P(λᵢ,t), controller 32 determines which of the signals generated by transducers 42 responsive to P(λᵢ,t) are generated responsive to photoacoustic waves originating at distances d from fiber end 38 corresponding to locations in bolus 22. (Distances d that correspond to bolus 22 are known from the location of blood vessel 23, which was determined as noted above.) From the signals responsive to photoacoustic waves 52 originating inside bolus 22 controller 32 determines values for P(λᵢ,d) for a plurality of locations in blood bolus 22 at different distances d from end 38. The controller uses the determined values for P(λᵢ,d) and equation (8) to determine a value for αₑ(λᵢ). Optionally the determined value for αₑ(λᵢ) is a best fit value that optimizes the fit of equation (7) to the determined values for P(λᵢ,d).

To determine the reduced scattering coefficient α'ₛ(λᵢ) for mensuration wavelength λᵢ, in some embodiments of the present invention, scattering of light in blood bolus 22 is measured at the wavelength. In some embodiments of the invention the scattering coefficient is determined from a wavelength dependent analytic function, i.e. a scattering coefficient function parameterized by at least one characteristic parameter. Optionally, the scattering coefficient function is determined assuming that scattering of light is substantially Mie scattering. As a result, as is known in the art, dependence of α'ₛ(λᵢ) on wavelength may be approximated by an expression of the form,

α'ₛ(λᵢ)=Bλᵢ^{-C}. (8)

In some embodiments of the present invention, values for the characteristic parameters B and C in equation (8) are determined for blood bolus 22 using methods known in the art, such as for example a method described in Mourant et al; “Mechanisms of Light Scattering from Biological Cells Relevant to Noninvasive Optical-Tissue Diagnostics”; Applied Optics Vol 37, issue 16, pg 3586-3593, June 1998. In addition, a reduced scattering coefficient α'ₛ(λ_{R}), hereinafter a “reference scattering coefficient”, for blood bolus 22 is determined for a reference wavelength λ_{R}. In terms of the reference wavelength and associated reference scattering coefficient, the reduced scattering coefficient α'ₛ(λᵢ) may be expressed by

α'ₛ(λᵢ)=α'ₛ(λ_{R})(λᵢ/λ_{R})^{-C}, (9)

In some embodiments of the present invention, a reference wavelength λ_{R} for a tissue region is a wavelength for which the absorption coefficient αₐ(λ_{R}) is known and the reference scattering coefficient α'ₛ(λ_{R}) is determined from equation (2) and measurements of an extinction coefficient αₑ(λ_{R}) at the reference wavelength. In some embodiments of the present invention, the absorption coefficient αₐ(λ_{R}) for a tissue region is known because the absorption coefficient of the tissue region is substantially determined by a component analyte whose concentration in the region is known. In some embodiments of the present invention, concentration of the component analyte is determined from a measurement of the extinction coefficient αₑ(λᵢ) for the region at a wavelength for which the extinction coefficient of the region is substantially equal to the absorption coefficient of the component analyte. Optionally, as described above, measurements of the extinction coefficient αₑ(λ_{R}) are acquired from time dependence of signals generated by transducers 42 responsive to photoacoustic waves stimulated in the region.

By way of a numerical example, for determining parameters of equation (9) required to determine α'ₛ(λᵢ) for blood, at 570 nm the magnitude of the reduced scattering coefficient α'ₛ(570) is between about 2 cm⁻¹ and about 3 cm⁻¹. The magnitude the absorption coefficient αₐ(570) of blood at 570 nm is about 280 cm⁻¹. The extinction coefficient αₑ(⁵⁷⁰) for blood at 570 nm is therefore substantially equal to the absorption coefficient α'ₐ(570) of blood. In addition, the absorption coefficient of blood at 570 nm is substantially equal to the absorption coefficient of hemoglobin. Furthermore 570 nm is an isobestic wavelength for hemoglobin at which the absorption cross-sections for oxygenated and deoxygenated hemoglobin are about equal. Therefore, at 570 nm the concentration of hemoglobin may be determined without having to know the ratio of oxygenated hemoglobin to total hemoglobin from a measurement of the photoacoustic effect at 570 nm. Equation (4) for blood at wavelength 570 nm becomes,

αₐ(570)=αₑ(570)=σₐₕ(570)xₕ, (10)

where σₐₕ(570) is the absorption coefficient for hemoglobin at 570 nm and xₕ is the concentration of hemoglobin in blood. In accordance with an embodiment of the present equation (10) provides a value for xₕ.

810 nm is another isobestic wavelength in the absorption spectrum of hemoglobin at which the absorption coefficient of blood is also dominated by the absorption coefficient σₐₕ(810) of hemoglobin. However, at 810 nm the reduced scattering coefficient is not negligible and equation (2) becomes,

αₑ(810)=[3σₐₕ(810)xₕ(σₐₕ(810)xₕ+π'ₛ(810))]^{1/2}. (11)

Since xₕ is known from equation (10), equation (11) may be solved to provide a value, in accordance with an embodiment of the present invention, for the reduced scattering coefficient α'ₛ(810) at 810 nm. The scattering coefficient α'ₛ(λᵢ) at wavelength λᵢ for blood may then be determined by using 810 nm for the reference wavelength λ_{R} and α'ₛ(810) for the reference scattering coefficient in equation (9) to provide,

α'ₛ(λᵢ)=α'ₛ(810)(λᵢ/810)^{-C}. (12)

To determine the coefficient C in equation (12), optionally, the extinction coefficient αₑ(λ) is determined for at least two other, non-isobestic, wavelengths of light at which hemoglobin concentration substantially determines the absorption coefficient of blood. Suitable wavelengths are preferably wavelengths that straddle 810 nm, for example 950 nm and 700 nm. Let the straddling wavelengths be represented by λ⁺ and λ⁻, and collectively by λ^{±}. Let the ratio of oxygenated hemoglobin to total hemoglobin in the blood be represented by S and the absorption cross sections for oxygenated and deoxygenated at wavelengths λ^{±} be represented by σ_{ahO}(λ^{±}) and σ_{ahD}(λ^{±}) respectively, then the absorption coefficient αₐₕ(λ^{±}) for hemoglobin in the blood at wavelengths λ^{±} is,

αₐₕ(λ^{±})=[σ_{ahO}(λ^{±})S+σ_{ahD}(λ^{±})(1−S)]xₕ. (13)

Using equations (2), (12) and (13) and the extinction coefficients αₑ(λ^{±}) determined for wavelengths λ^{±}, S and the exponent C may then be determined from the two equations,

αₑ(λ^{±})=[3αₐₕ(λ^{±})(αₐₕ(λ^{±})+α'ₛ(810)(λ^{±}/810)^{-C})]^{1/2}. (14)

Substituting the right side of equation (9) for α'ₛ(A) in equation (5) provides an equation of the form,

$${\sum }_{j}^{{N}_{A}} {σ}_{j} ( {λ}_{i} ) {x}_{j} = 1 / 2 \{ {α}_{s}^{′} ( {λ}_{R} ) {( {λ}_{i} / {λ}_{R} )}^{- C} + {[ {{α}_{s}^{′} ( {λ}_{R} )}^{2} {( {λ}_{i} / {λ}_{R} )}^{- 2 C} + ( 4 / 3 ) {{α}_{e} ( {λ}_{i} )}^{2} ]}^{1 / 2} \} . \qquad ( 15 )$$

Equation (15) for the N_{λ} mensuration wavelengths λᵢ provides a set of N_{λ} simultaneous equations in the unknown concentrations xⱼ, for each of which equations the right hand side the equation is known. In accordance with an embodiment of the invention, controller 32 provides a value for the concentration x₁ of glucose responsive to constraints on the concentrations xᵢ defined by the N_{λ} simultaneous equations. Optionally, since water is a major component of living tissue and since the concentration of water is relatively labile at least one of the mensuration wavelengths is a wavelength, for example 1350 nm, for which light is strongly absorbed by water and negligibly absorbed or scattered by other analytes in the body.

For a number of mensuration wavelengths N_{λ} equal to a number N_{A} of mensuration analytes, any of various well-known methods of manipulating and solving a set of simultaneous equations may be used to provide a value for x₁. In some embodiments of the present invention a number of mensuration wavelengths N_{λ} is greater than N_{A}, resulting in a number of simultaneous equations greater than the N_{A} unknown concentrations xⱼ. For such cases a suitable best-fit algorithm, such as a least squares algorithm may be used to provide a solution for concentrations xⱼ.

In some embodiments of the present invention, equation (15) is treated as an equation in (N_{A}+2) unknowns, where in addition to the unknown concentrations of the N_{A} mensuration analytes, the reference scattering coefficient α'ₛ(λ_{R}) and reference wavelength λ_{R} are considered to be unknown constants. Measurements of the extinction coefficient αₑ(λᵢ) are acquired for a plurality of N_{λ} mensuration wavelengths λⱼ equal to or greater than (N_{A}+2) to yield at least (N_{A}+2) simultaneous equations of the form of equation (15). A set of at least (N_{A}+2) simultaneous equation is sufficient to determine values for all concentrations xᵢ, as well as for α'ₛ(λ_{R}) and λ_{R}. Controller 32 provides a value for the concentration x₁ of glucose responsive to constraints on the concentrations xᵢ, reference scattering coefficient α'ₛ(λ_{R}) and reference wavelength λ_{R} defined by the N_{λ} simultaneous equations.

Similarly, in accordance with some embodiments of the present invention, the exponent “C” in equation (15), is also considered to be an unknown and measurements of αₑ(λᵢ) are acquired for at least (N_{A}+3) mensuration wavelengths λᵢ. The at least (N_{A}+3) extinction coefficient measurements yield at least (N_{A}+3) simultaneous equations of the form of equation (15). Controller 32 provides a value for the concentration x₁ of glucose responsive to constraints on the concentrations xᵢ, reference scattering coefficient α'ₛ(λ_{R}), reference wavelength λ_{R} and exponent C defined by the N_{λ}=(N_{A}+3) simultaneous equations.

In some embodiments of the invention for which the scattering coefficient is expressed as an analytic function having at least one unknown characteristic parameter which is a concentration of at least one of the mensuration analytes. Optionally, the concentration of at least one of the mensuration analytes includes the concentration of the target analyte. If the analytic function representing the scattering coefficient at wavelength λ is written S(λ,X), where X represents the set {xⱼ} of concentrations of the mensuration analytes or a subset thereof, then equation (5) becomes,

$${α}_{a} ( {λ}_{i} ) = {\sum }_{j}^{{N}_{A}} {σ}_{j} ( {λ}_{i} ) {x}_{j} = 1 / 2 \{ S ( {λ}_{i} , X ) + {[ {S ( {λ}_{i} , X )}^{2} + ( 4 / 3 ) {{α}_{e} ( {λ}_{i} )}^{2} ]}^{1 / 2} \} . \qquad ( 16 )$$

Similarly to the case of equation (5), the N_{λ} mensuration wavelengths provide a set of N_{λ} equations of the form of equation (16) in the N_{A} unknown concentrations xⱼ (1≦j≦N_{A}). The concentration of the target analyte is determined responsive to a solution of the set of equations.

For some target analytes and conditions it is possible to choose an advantageous set of mensuration wavelength for determining concentration of an analyte in accordance with a set of equations of the form of equation (5) or equation 16. For example, as noted above at 570 nm and 1350 nm the extinction coefficient for blood is substantially equal to the absorption coefficient of hemoglobin at 570 nm and water at 1350 nm respectively. At the isobestic wavelength 810 nm both the absorption coefficient and the reduced scattering coefficient contribute to the extinction coefficient for blood. It is possible and can be advantageous to assay glucose, in accordance with an embodiment of the invention, using these three wavelengths as mensuration wavelengths and hemoglobin, water and glucose as mensuration analytes.

In particular, at 810 nm it can be advantageous to use a set of simultaneous equation at the mensuration wavelengths for which at least one equation has the form of equation (16) and the reduced scattering coefficient is represented by an analytic function S(λᵢ,X). For example, if the concentrations of hemoglobin, water and glucose are represented by xₕ, x_{w} and x_{g}, respectively, S(λᵢ,X) in equation (16) optionally becomes S(λᵢ,xₕ,x_{w},x_{g}). Optionally, S(λᵢ,xₕ,x_{w},x_{g}) may be expanded in a Taylor series to a desired order in the concentrations xₕ, x_{w} and x_{g}. Coefficients in the Taylor series may be determined from a suitable model and/or empirically. For example, the coefficients may be determined using an expression for the reduced scattering coefficient described in “Dynamic optical coherence tomography in studies of optical clearing, sedimentation, and aggregation of immersed blood”; Valery V. Tuchin, Xiangqun Xu, and Ruikang K. Wang; APPLIED OPTICS Vol. 41, No. 1, 258-271, January 2002. Optionally, since at wavelengths 570 nm and 1350 nm the extinction coefficient is dominated by the absorption coefficient the reduced scattering coefficient is assumed to be zero and an expression for S(λᵢ,xₕ,x_{w},x_{g}) is used only in an equation of the form (16) at 810 nm.

In the above examples, extinction coefficients αₑ(λᵢ) for the N_{λ} mensuration wavelengths and for the reference wavelength that are used to determine glucose concentration x₁ are described as being determined using the photoacoustic effect. In some embodiments of the present invention optical coherence tomography (OCT) is used to determine at least one of the extinction coefficients used to determine concentration of an analyte.

OCT generally provides signals for determining an extinction coefficient having better SNR than photoacoustic effect signals at wavelengths for which the extinction coefficient is determined substantially by a reduced scattering coefficient. Photoacoustic effect signals generally have better SNR than OCT signals at wavelengths for which an extinction coefficient is dominated by an absorption coefficient. In accordance with an embodiment of the present invention, a glucometer for assaying glucose in a tissue region comprises at least one acoustic transducer and in addition an “OCT” interferometer. Photoacoustic signals generated by the at least one acoustic transducer are processed to determine extinction coefficients used to assay glucose for wavelengths at which an absorption coefficient dominates in determining a value for the extinction coefficient. Interference signals generated by the interferometer are processed to determine extinction coefficients used to assay glucose for wavelengths at which a scattering coefficient dominates in determining a value for the extinction coefficient.

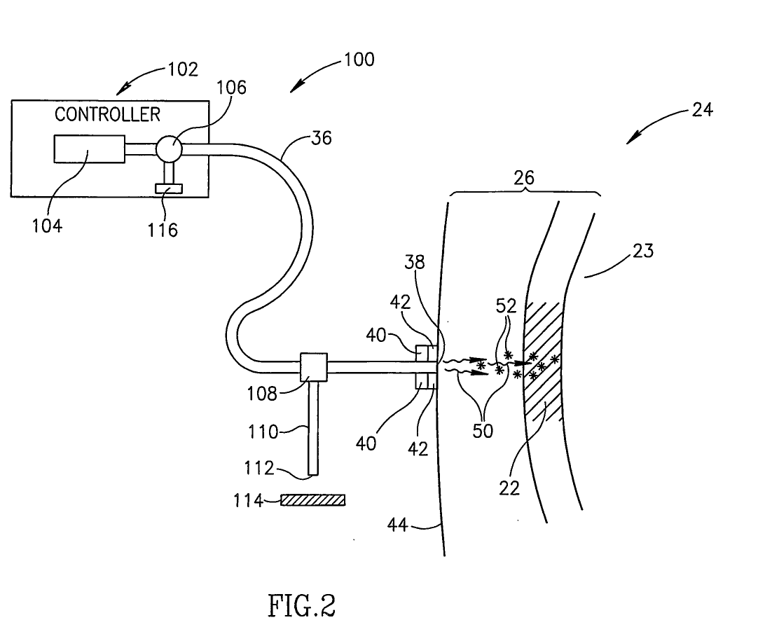

FIG. 2 schematically shows a glucometer 100, in accordance with an embodiment of the present invention comprising at least one acoustic transducer and an OCT interferometer. The components of the OCT interferometer are shown in a very schematic and simplified manner. Glucometer 100 is schematically shown assaying glucose in blood bolus 22 in blood vessel 23 of tissue region 26.

Glucometer 100 optionally comprises a controller 102, and a light source 104, optionally located in the controller, that provides semi-coherent light at wavelengths for which it is desired to determine an extinction coefficient to assay glucose, in accordance with the invention. An optic fiber 36 is coupled to light source 104 via an optical coupler 106. An end 38 of fiber 36 is optionally mounted to a support structure 40 to which acoustic transducers 42 are mounted.

To assay glucose in bolus 22 controller 102 controls light source 104 to transmit at least one pulse of light into optical fiber 36 at each of wavelength for which it is desired to determine an extinction coefficient for bolus 22. An optical coupler 108 couples a portion of light transmitted by light source 104 along optic fiber 36 to an optical fiber 110 and transmits a portion of the light towards end 38 of fiber 36 from which end the light exits the fiber to illuminate tissue region 24.

Some of the light that is transmitted along fiber 36 to exit the fiber at end 38 is absorbed in tissue region 26 and stimulates photoacoustic waves 52 in the region and some of the light is scattered by material in region 26. As in glucometer 20 acoustic transducers 42 generate signals responsive to photoacoustic waves 52. Some of the scattered light reenters fiber 36 at end 38 and propagates back towards controller 102 through optical coupler 108.

Light coupled to optical fiber 110 by coupler 108, exits the fiber from an end 112 and is reflected back into the fiber by a mirror 114. A portion of the light reflected back into optic fiber 110 is directed by optical coupler 108 to controller 102. When light reflected from mirror 114 and scattered light from tissue region 26 that reenters optic fiber 36 reaches controller 102, the light is directed by coupler 106 to a combiner 116. Combiner 116 superposes the scattered light and the reflected light at an interference region (not shown) to generate an interference signal. The position of mirror 114 relative to fiber end 112 is controlled by controller 102 to determine a desired path length from light source 104 to mirror 114 and back to combiner 116. The path length is determined so that substantially only light that is scattered in tissue region 26 from desired locations in the region generates an interference signal. Semi-coherent light source 104, couplers 106 and 108, optic fibers 36 and 110, mirror 114 and combine 116 cooperate to function as an OCT interferometer.

Controller 102 processes signals generated by acoustic transducers 42 to determine which of the signals correspond to photoacoustic waves originating at different locations in bolus 22. Controller 102 controls the position of mirror 114 to scan bolus 22 and generate interference signals corresponding to light reflected from different locations in the bolus.

For light at mensuration wavelengths having an extinction coefficient dominated by an absorption coefficient controller 102 optionally uses signals generated by transducers 42 corresponding to locations in bolus 22 to determine an extinction coefficient at the wavelength for the bolus. For light at mensuration wavelengths having an extinction coefficient dominated by a scattering coefficient, controller 102 optionally uses interference signals generated by combiner 116 corresponding to locations in bolus 22 to determine an extinction coefficient at the wavelength for the bolus. The controller uses extinction coefficients to assay glucose in the same way that glucometer 20 uses extinction coefficients to assay glucose.

In the description and claims of the application, each of the verbs, “comprise” “include” and “have”, and conjugates thereof, are used to indicate that the object or objects of the verb are not necessarily a complete listing of members, components, elements or parts of the subject or subjects of the verb.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons of the art. The scope of the invention is limited only by the following claims.

1. Apparatus for assaying a target analyte in a localized tissue region that may include the target and other analytes comprising:
a light source that illuminates the region with light at each of a plurality of wavelengths at which light is absorbed and/or scattered by tissue in the region wherein light at at least one of the wavelengths is absorbed or and/or scattered by the target analyte;
a signal generator that generates signals responsive to intensity of the light from the light source at different locations in the localized region; and
a controller that:
receives the generated signals;
processes the signals to determine an extinction coefficient for light in the localized region at each wavelength; and
determines the concentration of the target analyte responsive to a solution of a set of simultaneous equations having as unknown variables concentrations of a plurality of analytes in the region, one of which is the target analyte, wherein each equation in the set expresses a relationship between the extinction coefficient, the absorption coefficient and/or the reduced scattering coefficient for light at a different one of the plurality of wavelengths and at least one of the equations expresses a relationship between the extinction coefficient and the reduced scattering coefficient.

2. Apparatus according to claim 1 wherein the at least one equation that expresses a relationship between the extinction coefficient and the reduced scattering coefficient includes a dependence on the absorption coefficient.

3. Apparatus according to claim 1 wherein the reduced scattering coefficient at least one of the wavelengths is a measured value of the reduced scattering coefficient.

4. Apparatus according to claim 1 wherein the reduced scattering coefficient at least one of the wavelengths is a value determined responsive to an analytic expression.

5. Apparatus according to claim 1 wherein the reduced scattering coefficient at least one of the wavelengths is expressed as an analytic function.

6. Apparatus according to claim 5 wherein the analytic expression is a function of at least one unknown variable having a value determinable responsive to a solution of the simultaneous equations.

7. Apparatus according to claim 6 wherein the at least one unknown variable is a concentration of at least one of the target analyte and the other analytes.

8. Apparatus according to claim 4 wherein the function comprises an expression of the form Bλ^{-C} where λ represents the wavelength and B and C are constants.

9. Apparatus according to claim 1 wherein the signal generator comprises at least one acoustic transducer that generates signals responsive to acoustic energy that reaches the transducer from photoacoustic waves generated in the region by the light.

10. Apparatus according to claim 1 wherein the signal generator comprises an optical coherence tomography device that receives light from the light source that is scattered from the region and generates an interference signal responsive to an interference pattern between the scattered light and light from the light source reflected by a reflector.

11. Apparatus according to claim 1 wherein the controller identifies and locates the localized region in a larger region comprising the localized region.

12. Apparatus according to claim 11 wherein to identify and locate the localized region the controller:
controls the light source to illuminate the larger region with light that is absorbed by a component characteristic of the localized region;
receives signals generated by the signal generator responsive to intensity of the light from the light source in different locations in the larger region;
uses the signals to assay the characteristic component in different localized regions in the larger region; and
identifies and locates the localized region responsive to the assay.

13. Apparatus according to claim 11 wherein the apparatus comprises at least one acoustic transducer controllable to transmit ultrasound, and to identify and locate the localized region the controller:
controls the at least one transducer to transmit ultrasound into the larger region;
receives signals generated by the at least one acoustic transducer responsive to acoustic energy reflected by features in the larger region from the transmitted ultrasound; and
uses the signals to identify and locate the features and thereby the localized region.

14. Apparatus according to claim 1 wherein the localized region is a bolus of blood.

15. A method of assaying a target analyte in a region of body tissue that may include the target and other analytes comprising:
determining an extinction coefficient for light at each of a plurality of different wavelengths at which light is absorbed and/or scattered by tissue in the region and wherein light at least one of the wavelengths is absorbed and/or scattered by the analyte;
providing a value or an analytic expression for the reduced scattering coefficient at each wavelength; and
determining the concentration of the target analyte responsive to a solution of a set of simultaneous equations having as unknown variables concentrations of a plurality of analytes in the region, one of which is the target analyte, wherein each equation in the set expresses a relationship between the extinction coefficient, the absorption coefficient and/or the reduced scattering coefficient for light at a different one of the plurality of wavelengths and at least one of the equations expresses a relationship between the extinction coefficient and the reduced scattering coefficient.

16. A method according to claim 15 wherein the at least one equation that expresses a relationship between the extinction coefficient and the reduced scattering coefficient includes a dependence on the absorption coefficient.

17. A method according to claim 15 wherein determining the extinction coefficient at least one of the wavelengths of the plurality of wavelengths comprises:
from a given location illuminating the region with light at the wavelength so as to generate photoacoustic waves in the region;
determining a rate of decrease amplitude of the generated photoacoustic waves with increase of distance in the tissue region from the given location; and
determining the extinction coefficient from the determined rate of decrease.

18. A method according to claim 15 wherein determining the extinction coefficient at least one of the wavelengths of the plurality of wavelengths comprises:
from a given location illuminating the region with light at the wavelength;
using optical coherence tomography to determine a rate of decrease of intensity of the light with increase of distance in the tissue region from the given location; and
determining the extinction coefficient from the determined rate of decrease.

19. A method according to claim 15 wherein the reduced scattering coefficient at least one of the wavelengths is a measured value of the reduced scattering coefficient.

20. A method according to claim 15 wherein the reduced scattering coefficient at least one of the wavelengths is a value determined responsive to an analytic expression.

21. A method according to claim 15 wherein and comprising expressing the reduced scattering coefficient in at least one of the equations as an analytic function.

22. A method according to claim 21 wherein the analytic expression is a function of at least one unknown variable having a value determinable responsive to a solution of the simultaneous equations.

23. A method according to claim 22 wherein the at least one unknown variable is a concentration of at least one of the target analyte and other analytes.

24. A method according to claim 20 wherein the analytic expression comprises an expression of the form Bλ^{-C} where λ represents the wavelength and B and C are constants.

25. A method according to claim 15 and comprising identifying and locating the localized region in a larger region comprising the localized region.

26. A method according to claim 25 wherein identifying and locating the localized region comprises:
illuminating the larger region with light that is absorbed by a component characteristic of the localized region;
generating signals responsive to intensity of the light at different locations in the larger region;
using the signals to assay the characteristic component in different localized regions in the larger region; and
identifying and locating the localized region responsive to the assay.

27. A method according to claim 25 wherein identifying and locating the localized region comprises:
transmitting ultrasound into the larger region;
generating signals responsive to acoustic energy reflected by features in the larger region from the transmitted ultrasound; and
using the signals to identify and locate the features;
using the identities and locations of the features to identify and locate the localized region.

28. A method according to claim 15 wherein the localized region is a bolus of blood.
